(19) United States
(12) Patent Application Publication
(10) Pub. No.: US 2010/0248248 A1
(43) Pub. Date: Sep. 30, 2010

(54) Use of the Serological Assay of the Cytokine B-Lymphocyte Stimulator (Blys) as a Prognostic and Monitoring Test for Immune-Related Transfusion Reactions

(75) Inventors: Francesco CURCIO, Pagnacco (IT); Salvatore DE VITA, Udine (IT); Martina FABRIS, Udine (IT); Elio TONUTTI, Udine (IT); Cristina MELLI, Udine (IT)

Correspondence Address: PANITCH SCHWARZE BELISARIO & NADEL LLP, ONE COMMERCE SQUARE, 2005 MARKET STREET, SUITE 2200, PHILADELPHIA, PA, 19103, US

(73) Assignee: UNIVERSITA' DEGLI STUDI DI UDINE, Udine (IT)

(21) Appl. No.: 12/752,544

(22) Filed: Apr. 1, 2010

Related U.S. Application Data
(63) Continuation-in-part of application No. PCT/EP2008/063081, filed on Sep. 30, 2008.

(30) Foreign Application Priority Data
Oct. 1, 2007 (IT) UD2007A000183

(51) Int. Cl. C12Q 1/68; G01N 33/53

(52) U.S. Cl. 435/6; 435/7.92

(57) ABSTRACT

The use for the serum quantification of the cytokine B-Lymphocyte Stimulator (BLyS) for the evaluation of the risk of immunization and transfusion reactions after blood transfusion, and for the monitoring of patients undergoing blood transfusion or re-transfusion in a patient that includes an initial step of taking a sample of blood from the patient, a step of analyzing the blood sample to determine the concentration of cytokine BLyS, a step of comparing the BLyS levels determined in the previous step and one or more reference values of concentration of cytokine BLyS, a step of identifying a significant deviation between the determined concentration of cytokine BLyS and the reference values of concentration of cytokine BLyS indicated in the previous step and a step of assigning a risk and/or therapeutic effectiveness with respect to the immune-mediated transfusion reactions mentioned above, based on the previous steps.

### CROSS-REFERENCE TO RELATED APPLICATIONS

This application is a Continuation of International Application No. PCT/EP2008/063081, filed Sep. 30, 2008, which was published in the English language on Apr. 9, 2009, under International Publication No. WO 2009/043848 A2 and the disclosure of which is incorporated herein by reference.

### BACKGROUND OF THE INVENTION

The present invention concerns the use of the serological assay of the cytokine B-Lymphocyte stimulator (BLyS) as a prognostic and monitoring test for the clinical management of immunization and reactions following blood transfusions.

The cytokine B-Lymphocyte stimulator (BLyS), also known as “B-cell activating factor of the TNF family” (BAFF), was discovered and characterized in 1999 based on its homology with the members of the superfamily of the tumor necrosis factor (TNF) (Schneider P. et al. J Exp Med. 1999; 189(11): 1747-56 and Nardelli B. et al. Blood. 2001; 97(1):198-204).

BLyS plays a very important role in immune response, since it is now counted as one of the key factors in regulating B-cell development and differentiation (Mackay F., Browning J L. Nat Rev Immunol 2002; 2:465-75 and Batten M et al. J Exp Med 2000; 192(10): 1453-66).

BLyS is synthesized, expressed as a membrane protein and released in soluble form primarily by cells of the myeloid line such as monocytes, macrophages, neutrophils, dendritic cells (Huard B. et al. Int Immunol 2004; 16:467-475 and Nardelli B. et al Blood. 2001; 97(1):198-204).

Recently, the series of cell types able to secrete this cytokine has been further enlarged, including also non-myeloid cells, such as the cells of the medullar stroma (Gorelik L: et al. J Exp Med 2003; 198:937-945), synoviocytes (Ohata J. et al. J Immunol 2005; 174(2):864-70), astrocytes (Markus Krumbholz et al. J Exp Med. 2005; 201(2):195-200), the salivary gland epithelium (Ittah M, Miceli-Richard C., Eric Gottenburg J et al. Arthritis Res Ther. 2006; 8(2):R51) and the intestinal epithelium (Xu W., He B., Chiu A. et al. Nature Immunol 2007; 8(3):294-303).

BLyS exerts its function through interaction with three receptors, the most important of which, the BAFF receptor (BAFFR), is expressed in a peculiar manner by B lymphocytes (Ng LG et al. J Immunol. 2004; 173(2):807-17). The link between BLyS and BAFFR induces an increase in expression of several anti-apoptotic factors (Bcl2, Bcl-xL, Mcl-1), thus promoting mature B cell survival and proliferation (Craxton A, et al. J Exp Med. 2005; 202(10):1363-74).

BLyS has high homology with another member of the TNF superfamily called APRIL (A Proliferation Inducing Ligand) (Hahne M et al. J Exp Med 1998; 188:1185-1190), which shares with BLyS two of its three receptors, TACI (transmembrane activator and calcium-modulating cyclophilin ligand) and BCMA (B-cell maturation antigen) (Thompson J S, Schneider P, Kalled S L et al. J Exp Med. 2002; 192(1):129-35 and Seshasayee D, Valdez P, Yan Met al. Immunity 2003; 18(2):279-88).

The importance of BLyS in B-cell homeostasis has been brought to light by studies on murine models.

In BLyS knock-out mice, where BLyS expression has been abrogated, a profound alteration is observed of the pool of mature B lymphocytes (Gross JA et al. Immunity 2001; 15:289-302), whereas mice that hyperexpress BLyS (BLyS transgenic mice) develop many characteristics typical of autoimmune diseases.

These include spleno- and lymphoadeno-megaly, high serum levels of autoimmune-antibodies (rheumatoid factor, anti-DNA), B-cell infiltration of the parotid glands with subversion of the glandular architecture and loss of the secretory function, as is found in the course of Sjögren's syndrome (SS), renal alterations which greatly recall the glomerulonephritis typical of systemic lupus erythematosus (SLE) and finally they develop a B-cell neoplasia (Mackay F et al J Exp Med. 1999; 190(11):1697-710 and Thien M et al. Immunity 204; 20(6):785-98).

The experimental evidence therefore shows that, at physiological levels, BLyS promotes B-cell survival and differentiation (Mackay F, Browning J L. Nat Rev Immunol 2002; 2:465-75), but, at supraphysiological concentrations, it allows autoreactive B-lymphocytes to survive and proliferate, which would normally be suppressed by the immune system (Thien M et al. Immunity. 2004; 20(6):785-98).

In accordance with this experimental evidence, recently high serum and tissue levels of BLyS have been described in several autoimmune diseases as well as in IgA deficiency, all being characterized by an increased incidence of allergic reactions and blood transfusion reactions in general (Ramsey G et al. Transfusion 1995; 35:582-6; Rogers R L et al, Am J Hematol. 1998 April; 57(4):326-30).

Among the autoimmune diseases with increased BLyS levels, we can display: Sjögren's syndrome (SS), rheumatoid arthritis (RA), systemic lupus erythematosus (SLE), systemic sclerosis (SSc), multiple sclerosis (MS), mixed cryoglobulinemia (MC), celiac disease (CD), autoimmune thyroiditis (AIT) and Wegener's granulomatosis (Stohl W. B Curr Rheumatol Rep 2002; 4(4):345-50; Seyler T M et al J Clin Invest. 2005; 115(11):3083-92; Mariette X et al. Ann Rheum Dis. 2003; 62(2):168-71; Matsushita T et al. Arthritis Rheum. 2006; 54(1):192-201; M. Thangaraj et al. J Neuroimmunol 2004; 152:183-190; Fabris M et al. J Rheumatol 2007; 46:37-43; Fabris M et al. Scan J Gastroentherol 2007; 42(12):1434-9; Fabris M, et al. Autoimmun Rev. 2010 January; 9(3):165-9 and M. Krumbholz et al. J Autoimmun 2005; 25:298-302).

In general, in these autoimmune diseases, the serum and tissue levels of BLyS are correlated with the levels of disease-specific autoantibodies and the presence and level of lymphocyte infiltration in the affected tissues (synovial membrane, salivary glands) and in particular the formation of ectopic germinal centers appeared correlated with the presence of BLyS and APRIL (Jonsson M V et al J Clin Immunol 2005; 25:189-201, Szodoray P et al. Clin Immunol 2005; 17: 168-176).

Elevated BLyS levels have also been found in the course of organ rejection: in this context the BLyS/BAFFR interaction on T-lymphocytes promotes T cell activation and proliferation against the transplanted organ (Ye Q et al. Eur J Immunol 2004; 34: 2750-59). In a murine model of cardiac transplantation rejection due to MHC-mismatch, the blockade of BLyS-BAFFR can significantly extend the survival of the transplanted organs.

In addition, IgA deficiency (IgAD) is commonly associated with autoimmune disease and with allergic reactions, including transfusion reactions. And we also showed increased BLyS levels in patients with IgAD, independently from an associated autoimmune disorder. This result let us to hypothesize that the increased BLyS, and not only an associated autoimmune disorder, may represent a predisposing factor for immunization and immune-mediated transfusion reactions in such contest.

The immune-related transfusion reactions comprise all the possible complications following a blood transfusion, due to plasmatic or erythrocytic incompatibility, such as thrill-hyperthermic syndrome, allergic reactions, but most of all post-transfusion haemolytic reactions. Recently, it has been observed that autoimmune disease-affected patients present an increased production of irregular alloantibodies after blood transfusion compared to the general population (Ramsey G et al. Transfusion 1995; 35:582-6). Irregular alloantibodies are antibodies produced against non-self erythrocytic antigens after blood transfusions, pregnancy, active immunizations or passively acquired after immunoglobulin or plasma infusions or organ or bone marrow transplantations. The frequency of the presence of alloantibodies varies between 0.3 and 38% of the general population and is continuously growing, through the increased sensibility of the new methods used to detect alloantibodies in the blood. These alloantibodies are responsible for most of the haemolytic transfusion reactions with clinical relevance.

With such a background, one purpose of the present invention is to use the serological assay of cytokine B-Lymphocyte stimulator BLyS as a screening test for risk assessment, as a prognostic test for the prevention and clinical management of immunization and immune-related transfusion reactions (post-transfusion immunization, maternal-fetal incompatibility, transfusion reactions). The invention may be applied to all the patients candidate to transfusion or re-transfusion, with particular regard to patients with predisposing diseases (e.g., patients with autoimmune disease, IgAD, history of allergic reactions).

The invention will overcome the limits in the approach currently in use in the prevention and management of immunization and transfusion reactions after blood transfusion.

Another purpose of the present invention is to use the assay of cytokine BLyS as a method to identify patients where a personalized therapy to decrease or normalize BLyS levels (e.g., with corticosteroids or anti-BLyS agents under investigation, such as belimumab and atacicept) may be proposed, which will overcome the limits in the approach currently in use.

To overcome the drawbacks of the state of the art and to obtain these and other purposes and advantages, the Applicant has studied, tested and embodied the present invention.

### BRIEF SUMMARY OF THE INVENTION

The present invention is set forth and characterized in the independent claims, while the dependent claims describe other characteristics of the invention or variants to the main inventive idea.

The evidence regarding B cell population and the presence of BLyS was totally lacking in immune-related transfusion reactions here described. Thus, it never was hypothesized previously that BLyS could have an important pathogenic role also in immune-related transfusion reactions.

The formulation of this hypothesis has been possible putting together the relevant scientific background coming from the knowledge and the experience in systemic and organ-specific autoimmune diseases, in IgA deficiency, and in transfusion reactions, integrated with researches on BLyS in these disorders. This process came from the systematic integration among the different expertise of the co-inventors.

The results obtained by the inventors from preliminary studies in patients with or without transfusion reactions, have led the co-inventors to:

i) discover BLyS, at baseline and in the follow-up after transfusion, as a new useful marker for risk assessment and prognostic management of immunization (i.e. production of anti-blood cells antibodies) and therefore transfusion reactions after blood transfusions. In particular, BLyS serum levels, at baseline and in the follow-up after transfusion, appeared as a marker of risk and/or predisposition to immunization and therefore to transfusion reactions. Thus, serum BLlyS may identify patients at major risk of immunization and blood transfusion reactions, and in these patients blood transfusion should be further evaluated and avoided, whenever possible, if such an increased risk is present.

ii) identify BLyS assay, at baseline and in the follow-up after transfusion, as a method to detect patients where a personalized therapy to decrease or normalize BLyS levels (e.g., with corticosteroids or anti-BLyS agents under investigation, such as belimumab and atacicept) may be advisable. Thus, besides warning for transfusion (point i), BLyS serum levels assay may be a method to identify patients candidate to different treatment approaches, when transfusion can not be avoided.

In accordance with the above purposes, one feature of the present invention concerns the use of the serological assay of cytokine B-Lymphocyte stimulator (BLyS) as a method to identify patients with an increased risk of immunization and therefore of developing transfusion-related immune-mediated diseases, such as post-transfusion immunization, maternal-fetal incompatibility, transfusion reactions;

in all patients who are candidate to blood transfusion or underwent blood transfusion with or without symptoms or signs suggestive for transfusion reactions, with particular regard to patients who are at major risk of transfusion reactions (i.e. autoimmune diseases, immunodeficiency);

in a method which comprises the following steps:
a first step of taking a blood sample from the patient from which the serum can be obtained by centrifugation, the sample being taken at baseline (i.e., before transfusion, and after approximately 1 and 4 months post-transfusion);
a second step of examining the samples of serum (i.e., before transfusion, and after approximately 1 and 4 months post-transfusion) in order to determine the concentration of cytokine BLyS, typically by using commercial kits;
a third step of comparing the concentration of cytokine BLyS determined in the second phase and the reference values of concentration of cytokine BLyS previously obtained on a healthy population;
a fourth step of identifying a significant deviation, deriving from the comparison in the third step, between the concentration of cytokine BLyS determined in the second phase and the reference values of concentration of cytokine BLyS previously obtained on a healthy population control, so to select the patients at risk of said diseases (transfusion reactions);
a fifth step of comparing the concentration of cytokine BLyS determined in the second step for the selected patients and one or more reference values of concentration of cytokine BLyS; these values are in a range of benchmarks to BLyS previously obtained on a series of patients with transfusion-related immune-mediated diseases.
a sixth step of assigning an increased risk of developing one or more determinate diseases from among the immune-mediated diseases as above, according to the comparison made in the fifth step.

The present invention advantageously uses in the second step, for the analysis of the concentration of BLyS in the patients' serum, an automated apparatus able to perform immune-enzymatic assays (Enzyme-Linked Immunosorbent Assay: ELISA), of the type usually present in the largest hospital analyses labs, without needing substantive modifications to the plants or the organizational structures of the wards concerned.

Another innovative feature of the present invention is the use of cytokine B-lymphocyte Stimulator (BLyS) as a marker to identify patients where a personalized therapy to decrease or normalize BLyS levels (e.g., with corticosteroids or anti-BLyS agents under investigation, such as Belimumab or Atacicept) may be proposed.

To this purpose it is possible to advantageously use the monitoring of the serological concentration of BLyS after blood transfusion as a marker of transfusion reaction predisposition if the serum levels of BLyS overcome those included in the normal range, or to use the assay of BLyS as a marker in a method to warn for transfusion/re-transfusion or control the effectiveness of therapeutic treatments of patients undergoing blood transfusions, that comprises the following steps:

a first step of taking a sample of blood from the patient to obtain serum before the new therapy;

a second step of examining the serum sample to determine the concentration of cytokine BLyS;

a third step of taking a sample of blood from a patient at fixed times after the blood transfusion or after start of the anti-BLyS therapy (for example: 1 and 4 months after blood transfusion);

a fourth step of examining the sample/samples of blood taken in the third step, to determine the concentration of cytokine BLyS;

a fifth step of comparing the concentration of cytokine BLyS determined in the second step and those determined in the fourth step;

a sixth step of identifying a significant deviation, deriving from the comparison in the fifth step, between the concentration of cytokine BLyS determined in the second step and the values of the concentration of cytokine BLyS obtained in the fourth step;

a seventh step of attributing a risk of immunization and therefore of developing transfusion reaction or to test the efficacy of anti-BLyS therapies for the prevention of immunization and transfusion reactions to the deviation identified in the sixth step.

The present invention allows to improve the diagnostic/prognostic approach and the therapeutic monitoring of patients undergoing blood transfusions.

A variant of the present invention provides that the use of the BLyS assay according to the present invention can be integrated with the following analyses:

physical and biochemical examination of the patient, based on the best current knowledge, to identify possible additional risk factors for immunization as transfusion reactions after blood transfusion/re-transfusion, as well as the main signs and symptoms of the transfusion reactions;

molecular analyses on DNA extracted by the peripheral blood taken from the patient (genetic predisposition linked or not to BLyS expression).

### BRIEF DESCRIPTION OF THE SEVERAL VIEWS OF THE DRAWINGS

The foregoing summary, as well as the following detailed description of the invention, will be better understood when read in conjunction with the appended drawings. For the purpose of illustrating the invention, there are shown in the drawings embodiments which are presently preferred. It should be understood, however, that the invention is not limited to the precise arrangements and instrumentalities shown.

These and other characteristics of the present invention will become apparent from the following description of a preferential form of embodiment, given as a non-restrictive example with reference to the attached drawings wherein in the drawings:

FIG. 1 is a graph comparing serum B-Lymphocyte Stimulator (BLyS) levels in celiac patients (CD) with respect to healthy blood donors (HBDs) [range of normality: <1.145 ng/ml, mean+2SD];

FIG. 2 is a graph showing the significant correlation between the concentration of cytokine B-Lymphocyte stimulator (BLyS) and the concentration of antibodies a-tTG in celiac patients;

FIG. 3 is a graph showing the significant reduction of B-Lymphocyte stimulator (BLyS) concentration following the gluten-free diet (GFD) in celiac patients (from 1.619±0.410 ng/ml to 1.283±0.310 ng/ml; *p=0.0122, Wilcoxon signed rank test);

FIG. 4 is a graph comparing serum B-Lymphocyte Stimulator (BLyS) levels in IgAD patients, globally (IgAD tot) and when distinguished in 2 subgroups: IgAD with celiac disease (IgAD+CD) and without CD (IgAD) with respect to healthy blood donors (HBDs) [range of normality: <1.145 ng/ml, mean+2SD];

FIG. 5 is a graph comparing serum BLyS levels in patients with autoimmune thyroiditis (AITD) globally and when distinguished between Hashimoto's thyroiditis (HT) and Graves/Basedow's disease (GBD) with respect to healthy blood donors (HBDs) [range of normality: <1.145 ng/ml, mean+2SD];

FIG. 6 is a graph comparing serum BLyS levels in HT patients with normal or reduced (hypo) FT4 levels;

FIG. 7 illustrates BLyS serum levels in 34 random patients candidates to blood transfusion (BT), in 22 patients who did not develop immune-mediated transfusion reactions post blood transfusion (No BTR) and in 21 patients who developed immunization or transfusion reactions post blood transfusion (Yes IMTR) compared to 77 age-sex matched healthy blood donors. Overall, serum BLyS levels in patients candidates to BT were superimposable to those in HBDs (0.78±1.01 ng/ml vs 0.66±0.24 ng/ml; p=ns) even if we could find some cases with very high BLyS levels. Patients who underwent BT and did not develop IMTR presented significantly higher BLyS levels than HBDs (1.41±0.94 ng/ml; p<0.0001), while patients who underwent BT and developed IMTR tended to present even higher BLyS levels (2.33±2.23 ng/ml; p<0.0001 vs HBDs and p=ns vs No IMTR). Statistical analyses were done using the Mann Whitney non parametric t-test); and

FIG. 8 illustrates baseline and post-transfusion BLyS serum levels in a subgroup of patients who were assayed before blood transfusion (BT) and after 15.6±8.5 days. Overall, a significant increase of serum BLyS levels was found from baseline and post-transfusion samples (0.64±0.40 ng/ml vs 1.32±0.69 ng/ml, p=0.0098 by Wilcoxon signed rank test). No data were available about immunization of these patients since time after BT was not enough to evaluate the development of anti-blood cells antibodies and no patients have developed TR. Anyway BT per se generally seems to determine a early significant increase of serum BLyS levels.

### DETAILED DESCRIPTION OF THE INVENTION

The present invention takes as its base what is known in the state of the art regarding cytokine B-Lymphocyte stimulator (BLyS) to perfect an innovative use of the serological assay this cytokine for the prevention and management of immunization after blood transfusion and immune-mediated transfusion reactions.

In particular, the experimental results which Applicant has obtained concern the expression and role of BLyS in the following conditions:

celiac disease;

IgA deficiency;

autoimmune thyroiditis;

immune-mediated post-transfusion immunization, maternal-fetal incompatibility, transfusion reactions; characterized by an immune-mediated response (production of alloantibodies, i.e., immunization, with or without any specific clinical sign and symptom of blood transfusion reactions, i.e., transfusion reaction) responsible for specific organic symptoms, by autoantibody secretion, by a strong association among them and with other autoimmune diseases and by an increased risk of developing B or T cell clonality, have led to identify and propose BLyS as a new diagnostic, prognostic and therapeutic marker in these pathologies.

Furthermore, based on the results obtained in post-transfusion immunization, maternal-fetal incompatibility, transfusion reactions, the present invention can be extended to all the other immune-mediated transfusion reactions where BLyS may be identified in future.

The previous published results of the Applicant with the study of BLyS in rheumatoid arthritis (RA), Sjogren's syndrome, mixed cryoglobulinemic syndrome (MC), autoimmune thyroiditis, celiac disease and IgA deficiency, have allowed the Applicant, for the first time since BLyS cytokine was discovered, to investigate the probable key role of BLyS expression in post-transfusion immunization, maternal-fetal incompatibility, transfusion reactions. Thus, previous published researches and discoveries for what concerns BLyS overexpression in autoimmune diseases and immunodeficiency, which are characterized by an increased risk of immunization and transfusion reactions, were a pre-requisite for the present discovery.

In RA, the Applicant confirmed previous studies demonstrating increased serum BLyS levels in about 20% of RA patients, and that these BLyS levels were greatly increased after treatment of RA patients with a monoclonal antibody producing CD20+ B-cell depletion (rituximab) (Quartuccio L et al. Reumatismo 2004; 56(3):143-6; M. Fabris et al, Ann Rheum Dis 2009; 68 (Suppl3):75).

In MC syndrome and viral infection by the hepatitis C virus (HCV), the Applicant has shown that HCV infection per se is able to induce an increased expression of BLyS and contributes, in a subset of predisposed subjects, to sustain the autoreactive B cell proliferation, favouring the appearance of the cryoglobulinemic syndrome. The development of the syndrome coincided with a further increase in the expression of BLyS, since the values of serum BLyS in patients with cryoglobulinemic syndrome were significantly higher than in subjects with only chronic HCV infection (Fabris M et al, J Rheumatol 2007; 46:37-43). Overall, anti-BLyS therapy appeared for the first time as an option in MC syndrome based on these novel data.

With these premises, the Applicant has hypothesized that there could be an increased level of BLyS also in celiac disease and autoimmune thyroiditis.

This effect would seem mainly mediated by the endogen antiviral response, that is, interferon, which various studies have shown to be in vitro a powerful inductor of BLyS expression.

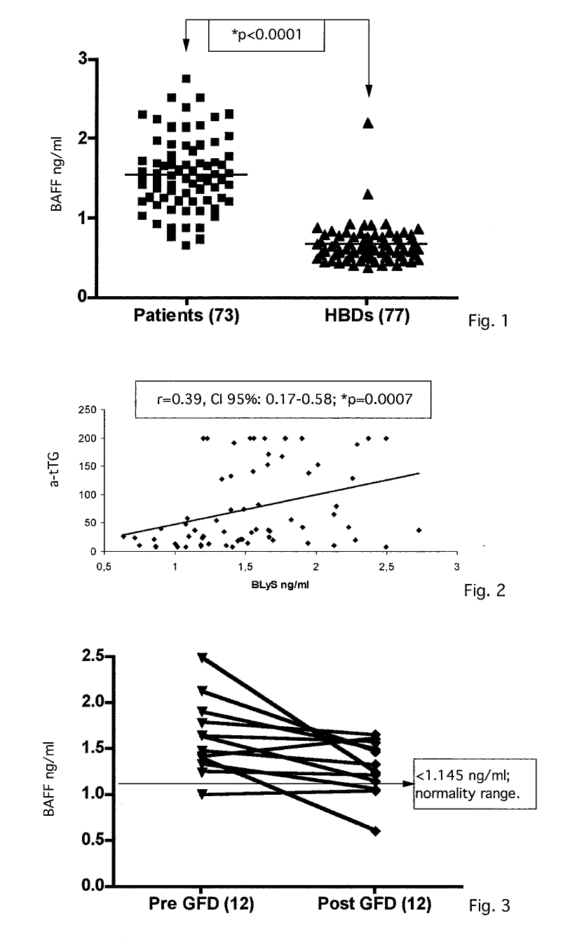

The role of B lymphocytes in the pathogenesis of celiac disease has so far appeared marginal compared to the fundamental role played by HLA and T lymphocyte activation (LM Sollid, Thorsby E. Gastroenterology 1993; 105: 910-22, Spurkland A, et al. Tissue antigens 1997; 49: 29-34 and Molberg Ø, et al. Gastroenterology 2003; 125:337-44). However, Applicant has shown that BLyS is found at high serum levels in a high percentage (>80%) of patients affected by celiac disease (FIG. 1).

In particular, FIG. 1 shows the serum levels of BLyS in celiac patients versus healthy controls (HBDs, consisting of healthy subjects, blood donors, comparable in age and sex to the patients in the study). The serum levels of BLyS are significantly higher in celiac patients compared with the healthy control population (Mann Whitney t-test, *p<0.0001). [range of normality: <1.145 ng/ml, mean+2SD].

This result, never shown before, proposes a new key in the interpretation of the pathogenesis of this widespread (about 1.1% of the population) enteropathy. Of particular importance is that, compared with all the autoimmune disorders where BLyS has been studied until now, in celiac disease the up-regulation of BLyS is the widest ever recorded, since it concerns 4 patients out of 5. Furthermore, the serum levels of BLyS measured and analyzed by Applicant show a significant correlation with those of the specific-disease antibodies, the anti-transglutaminase (a-tTG) (FIG. 2, which shows the correlation between serum levels of BLyS and levels of a-tTG IgA, Spearman rank test: r=0.399, 95% CI: 0.1724-0.5856, p=0.0007), and both BLyS and a-tTG diminish in concurrence with the introduction of the gluten-free diet and clinical remission of the disease (FIG. 3, which shows the modulation of the serum levels of BLyS after a gluten-free diet). A general significant reduction can be seen in the BLyS levels after the diet (from 1.619±0.410 ng/ml to 1.283±0.310 ng/ml; *p=0.0122, Wilcoxon signed rank test), even though 8/12 (75%) of the patients still have post-diet BLyS levels above the range of normality (>1.145 ng/ml, assay with ELISA kit R&D Systems Quantkine ELISA kit, Minneapolis, 55413 USA).

However, even in those cases which become a-tTG negative and which reach clinical remission, the BLyS levels, although substantially reduced, do not reach the range of normality, suggesting the persistence of a sub-clinical state of disease or a higher base level of BLyS predisposing to the disease and widely shared, equal to the HLA genotype.

Recent evidence of a possible infective co-participation (Rotavirus) in the pathogenesis of celiac disease provides another hypothesis of the raising of the BLyS serum levels, similarly to what was observed in the cryoglobulinemic syndrome.

The BLyS assay could therefore represent an additional diagnostic tool in cases of doubt, with an atypical presentation or with negative serum levels of a-tTG, or where the intestinal biopsy is precluded or not ethically indicated or again as screening in classes of individuals at greater risk of developing the disease.

Furthermore, the persistence at a systemic level of high serum levels of BLyS could contribute to the development of other autoimmune diseases in genetically predisposed individuals, just as, thanks to its powerful anti-apoptotic effect, BLyS would promote further genetic mutations in the expanded B cells until escape from the initial trigger and generation of a clonal population.

In the 66 patients with mixed cryoglobulinemic syndrome previously analyzed by the Applicant, the only clinical feature that showed a significant association with higher levels of BLyS was the presence of a clonal B cell proliferation: patients with a B cell clonality had a percentage of subjects with very high levels of BLyS significantly higher compared to patients without B cell clonality (33.3% versus 9.8%; OR=4.6, CI=1.12-18.96, p=0.04).

In these cases one might think that, as shown in some B cell neoplasms (Hodgkin's and non-Hodgkin's lymphoma, multiple myeloma, chronic lymphocytic leukemia) and more recently in B lymphocytes infiltrating the salivary glands of patients with primary SS (C Daridon et al. Arthritis Rheum 2007; 56:1134-44), B cell clones may also secrete BLyS and contribute to the disease, by a mechanism of autocrine stimulation.

BLyS, as previously shown in the course of cryoglobulinemia and SS and in several neoplastic disorders, could play an important role in the multi-step process which leads to the development of the lymphoma in celiac disease too, in fact it can stimulate both B and T cells (Mackay F, Leung H. Semin Immunol. 2006; 18(5):284-9). Applicant's finding of a very high BLyS level (8.5 ng/ml) in a celiac patient with a diffuse large B cell intestinal lymphoma is in accordance with this hypothesis.

This result therefore suggests a possible use of the BLyS assay as a diagnostic support in cases of a celiac patient where a B/T cell clonality is suspected (persistence of high a-tTG levels despite the strict adherence to the gluten-free diet).

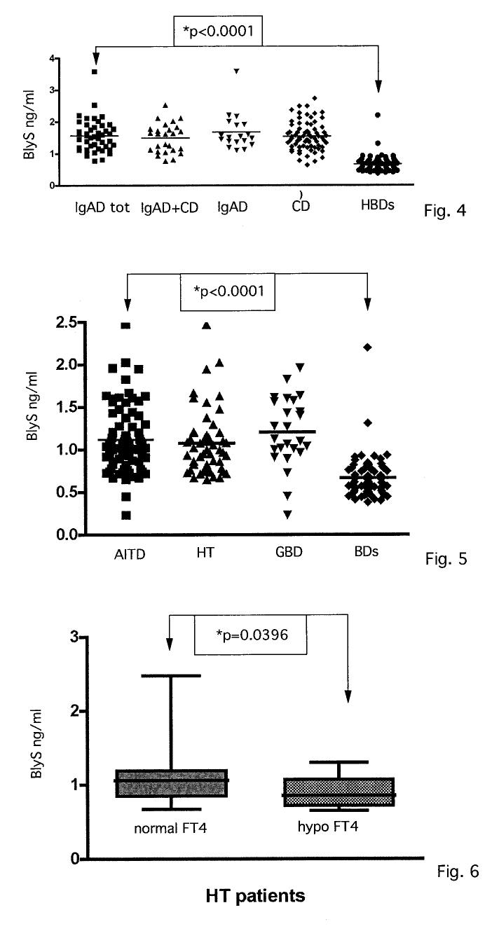

Selective primary IgA deficiency (IgAD) is the most common form of immunodeficiency, with an estimated incidence at 1:600 in Caucasians. Individuals with isolated IgAD have normal IgA genes, but have a defect of terminal lymphocyte differentiation, which leads to underproduction of serum and mucosal IgA (Cunningham-Rundles C. J Clin Immunol 2001; 21(5):303-9). There have been many diseases reported in association with IgAD, such as allergies, gastrointestinal tract and recurrent upper respiratory tract diseases and, in particular, autoimmune diseases (Liblau R S et al. Int Arch Allergy Immunol 1992; 99(1):16-27). The most common association is with celiac disease (CD), which has special significance since CD is usually diagnosed by detection of specific IgA antibodies, which are obviously lacking in IgAD patients. As illustrated in FIG. 4, Applicant discovered that BLyS serum levels are significantly more elevated in IgAD patients (1.57±0.51 ng/ml) than in controls (0.66±0.24 ng/ml; p<0.0001). In particular, 77.8% (35/45) of IgAD patients have BLyS levels over the range of normality (>1.14 ng/ml). Among the IgAD patients analyzed, 26 were affected by celiac disease CD but they did not differ significantly from the 19 patients with IgAD and without celiac disease CD (1.49±0.46 ng/ml versus 1.67±0.57 ng/ml, p=ns). No difference was found between BLyS levels in IgAD patients and the previously described series of patients with CD and normal IgA, (1.54±0.46 ng/ml). Thus, BLyS is upregulated in patients with IgAD, and could be one of the factors in the strong association between IgAD and autoimmune diseases, but also in the increased risk of developing B cell clonality. The present invention makes innovative use of the BLyS assay as a prognostic marker of the development of B cell clonality in subjects affected by IgAD.

Autoimmune thyroid diseases (AITD) are common autoimmune diseases, affecting up to 5% of the general population, with females affected more than males. Thyroid-directed autoimmunity is manifested in two classical autoimmune conditions: Hashimoto's Thyroiditis (HT) resulting in hypothyroidism (anti-TPO and anti-Thyroglobulin) and Graves-Basedow's disease (GBD) resulting in hyperthyroidism (TSH-Receptor agonist autoantibodies). AITD are frequently associated with other autoimmune diseases (celiac disease, type 1 diabetes mellitus, systemic connectivitis). Probably they share a common autoimmune-prone phenotype. Like other autoimmune diseases, AITDs present an increased risk of developing B-cell clonal diseases (especially HT patients).

As illustrated in FIG. 5, Applicant has studied a series of 77 Caucasian patients with AITD, 10M/67F, mean age 48.2±16.1, 52 with HT and 25 with GBD, and analyzed BLyS serum levels compared to 77 age/sex matched healthy controls. AITD patients showed a significant increase of BLyS levels (p<0.0001), GBD patients tended to have higher BLyS than HT (p=0.06). No significant correlation was found between BLyS levels and autoantibodies, both in HT and in GD. In contrast, a positive correlation was found between BLyS and FT4 (r=0.31; p=0.012), while an inverse correlation was found with TSH (r=−0.45; p=0.0002).

In fact, in HT patients BLyS is significantly more elevated in patients with normal FT4 levels than in patients with hypothyroidism (*p=0.0396) (FIG. 6).

In the present invention, for the first time, Applicant hypothesized and found elevated BLyS levels in AITD patients, suggesting an important pathogenetic role of BLyS also in these autoimmune disorders. In an innovative manner than previously demonstrated in systemic autoimmune diseases (RA, SS, LES), levels of BLyS correlate with thyroid functionality, but not with autoantibodies secretion. BLyS is therefore higher in the first euthyroideal phase of HT, and correlates with the level of hyperthyroidism in GBD, as a marker of gland activation, but not of plasma cells autoantibody secretion; it decreases when the gland loses its function, clinically manifested by hypothyroidism. Moreover, BLyS overexpression may represent one of the possible mechanisms explaining the increased percentage of AITD patients developing other autoimmune or lymphoproliferative diseases.

Thus, the present invention suggests using BLyS serological assay for the diagnosis, prognosis and screening of treatment efficacy in AITD patients.

It has been observed recently that patients with autoimmune diseases have a greater tendency to produce irregular alloantibodies after blood transfusion than the general population. At present, there are no known markers that can predict the development of transfusion reactions. The association with autoimmune diseases and the immune-mediated mechanism led the Applicant to consider a possible role of cytokine BLyS also in transfusion reactions. To this aim, a pilot study was conducted on 5 patients from the Blood Products Distribution Laboratory of Udine University Hospital: 2 patients (pts Type A) that despite repeated transfusions had never demonstrated the development of allo/autoantibodies; and 3 patients (pts Type B) who had developed allo/autoantibodies after transfusion of multiple units of concentrated red blood cells. In patients with allo/autoantibody reactions (Type B) the levels of BLyS/BAFF tended to be higher than in patients without reactions (Type A), (average 2.48 ng/ml versus 1.29 ng/ml). In addition, all type B patients showed BLyS levels above the threshold of normality (>1.14 ng/ml).

In Sjogren's syndrome, which shows concomitant MC syndrome in 5-10% of cases, the Applicant demonstrated high serum and tissue BLyS levels that are produced also by parotid gland epithelial cells and may be responsible for B-cell proliferation and resistance to anti-B cell therapy with rituximab (Quartuccio L et al. Open Rheumatol J 2008; 2:38-43).

The Applicants also showed, for the first time in the medical literature, significantly increased levels of BLyS in celiac disease and autoimmune thyroiditis (Fabris M et al. Scan J Gastroentherol 2007; 42(12):1434-9; Fabris M, et al. Autoimmun Rev. 2010 January; 9(3):165-9), two additional autoimmune diseases characterized by an increased risk of immunization and transfusion reactions. Finally, the Applicant also discovered a significant increased of serum BLyS levels in IgAD (Fabris M et al. Ann N Y Acad Sci. 2009 September; 1173:268-73), strongly linked to autoimmunity and transfusion reactions (Rogers R L et al, Am J Hematol. 1998 April; 57(4):326-30). It has been observed recently that patients with autoimmune diseases have a greater tendency to produce irregular alloantibodies after blood transfusion than the general population (Ramsey G et al. Transfusion 1995; 35:582-6).

At present, there are no known markers that can predict the development of transfusion reactions. The increased frequency of transfusion reactions in autoimmune diseases, and the immune-mediated mechanism of most transfusion reactions, led the Applicant to consider a possible role of cytokine BLyS also in transfusion reactions.

To this aim, a pilot study was conducted on 5 patients from the Blood Products Distribution Laboratory of Udine University Hospital: 2 patients (pts Type A) that despite repeated transfusions had never demonstrated the development of allo/autoantibodies; and 3 patients (pts Type B) who had developed allo/autoantibodies after transfusion of multiple units of concentrated red blood cells. In patients with allo/autoantibody reactions (Type B) the levels of BLyS/BAFF tended to be higher than in patients without reactions (Type A), (average 2.48 ng/ml versus 1.29 ng/ml). In addition, all type B patients showed BLyS levels above the threshold of normality (>1.14 ng/ml).

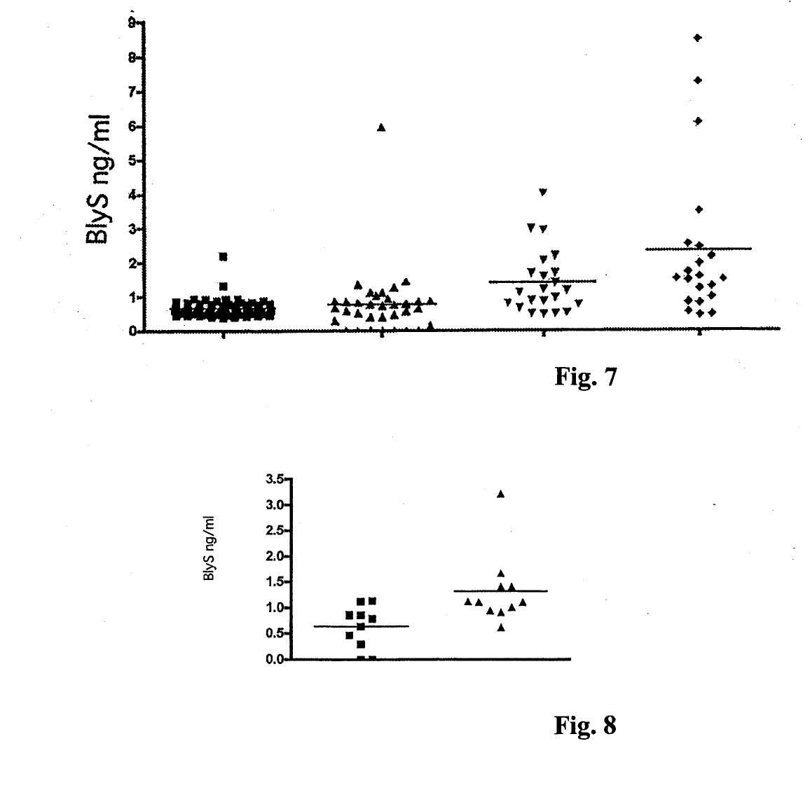

These preliminary results led the Applicant to further extend the study (FIG. 7) and BLyS serum levels were analysed in 34 randomly selected patients candidates to blood transfusion (BT) but not transfused, in 22 patients who did not develop neither immunization nor transfusion reactions post blood transfusion (No BTR), and in 21 patients who developed immunization (n. 19) or transfusion reactions due alloantibodies (n. 2) post blood transfusion (Yes IMTR). Data were always compared to a previously published series of 77 age-sex matched healthy blood donors. Overall, serum BLyS levels in patients candidates to BT appeared superimposable to those found in HBDs (0.78±1.01 ng/ml vs 0.66±0.24 ng/ml; p=ns). Patients who underwent BT and did not develop IMTR presented significantly higher BLyS levels than HBDs (1.41±0.94 ng/ml; p<0.0001), while patients who underwent BT and developed IMTR tended to present even higher BLyS levels (2.33±2.23 ng/ml; p<0.0001 vs HBDs and p=ns versus no IMTR). Of note the patient with the highest BLyS levels developed two severe transfusion reactions to two different blood bags. Among patients with IMTR there were 15/21 (71.4%) of cases with BLyS serum levels over the range of normality (>1.14 ng/ml), while among patients without IMBT there were 12/22 (54.5%) of cases with BLyS serum levels over the range of normality (OR=2; p=ns).

Statistical analyses were done using the Mann Whitney non parametric t-test for mean comparison and the Fisher's exact test for contingency tables.

We then performed BLyS serum quantification in repeated samples from the corresponding patient, before blood transfusion and 15.8±8.5 days after tranfusion, confirming (FIG. 8) that BT per se very often (77%) determines a significant increase in serum BLyS levels (0.64±0.40 ng/ml vs 1.32±0.69 ng/ml, p=0.0098 by Wilcoxon signed rank test), and always if the repeated test was performed within one month after transfusion (only two patients showed unchanged BLyS levels, and these two patients were the only ones where the blood sample was collected after more than one month after transfusion).

Thus, results indicated that BT is associated with an increase in BLyS serum levels, and such an increase appeared higher in patients who develop immunization after transfusion (a condition at higher risk to progress to a frank transfusion reaction) or an immune-mediated transfusion reaction (antibodies to blood cells plus clinical sign and symptoms of transfusion reaction).

The present invention therefore provides, in an innovative manner, the assay of serum BLyS in the following situations:

i) risk of immunization and of transfusion reactions (post-transfusion immunization, maternal-fetal incompatibility, transfusion reactions) after blood transfusion and selection of patients at higher risk of immunization and transfusion reactions where transfusion should be re-evaluated for its indications, or where a dedicated treatment before and/or after blood transfusion is advisable.

Patients with autoimmune diseases, or with immunological deficiency such as IgA deficiency and common-variable immunodeficiency, are identified as a subset of at even higher risk, due to published data, including data recently published by the Applicant, about increased BLyS expression in these settings.

The present invention applied to the prediction and/or screening of effective therapies in immune-mediated transfusion reactions in a patient therefore comprises the following steps:

a step of taking a sample of blood from which to obtain the patient's serum;

a step of examining the serum sample to determine the concentration, or assay, of cytokine BLyS, using the ELISA technique;

a step of comparing the concentration of cytokine BLyS determined in the previous step and one or more reference values of concentration of cytokine BLyS, which values may be those determined on a healthy population (healthy blood donors: HBDs) or a population of patients with a certain diagnosis of immune-mediated transfusion reaction or on a sample of serum from the same patient analyzed before blood transfusion or before the initiation of anti-BLyS therapy;

a step of identifying a significant deviation, deriving from the previous step, between the determined concentration of cytokine BLyS and the reference values of concentration of cytokine BLyS indicated in the previous step;

a step, of the decisional-deductive type, so as to assign a risk and/or prognosis regarding a particular clinical manifestation (production of alloantibodies, transfusion reactions), or to a level of therapeutic effectiveness in the course of immune-mediated diseases mentioned above, according to the previous steps.

In the particular case of the diagnostic method, the comparison step is carried out between the concentration of cytokine BLyS determined in the patient and one or more reference values of concentration of cytokine BLyS determined on a healthy population. According to this, from the step of identifying a significant deviation we select the patient as affected by one of said above-mentioned pathological conditions. Moreover, between the step of identifying a significant deviation and the last step of the decisional-deductive type, we have a further comparison step, between the concentration of cytokine BLyS determined in the patient and the values of concentration of cytokine BLyS of a population of patients with a certain diagnosis of immune-mediated disease and/or the presence of a particular clinical manifestation, so as to assign, in the last step, a risk of a determined immune-mediated disease from among all the above-mentioned immune-mediated diseases.

Moreover, the adoption of this assay does not entail substantive modifications to the plants or organizational structures of the wards involved in using this new marker, since the assay is effected with the ELISA technique using an automated apparatus commonly present in the major hospitals.

The present invention therefore provides, in a innovative manner, the use of BLyS assay also in the following situations:

ii) prognostic marker for post-transfusion immunization, maternal-fetal incompatibility, transfusion reactions in the general population;

iii) prognostic marker for post-transfusion immunization, maternal-fetal incompatibility, transfusion reactions in conditions predisposing to immunization and to blood transfusion reactions, such as autoimmune diseases and immunological deficiency (IgA deficiency, common variable immunodeficiency);

iv) monitoring patients undergoing blood transfusions (marker of activation of the immune system, prevention of transfusion reaction, effectiveness of therapy, etc.);

in these cases, based on the present invention, the method of monitoring comprises the same steps as the above described diagnostic method, applied to a patient with an autoimmune disease during his clinical follow-up with or without treatment, in which, in the third phase, the comparison may also be made with one or more values of cytokine BLyS concentration previously detected in the patient and where on the basis of the fifth step of diagnosis, in a subsequent sixth step it may be decided to repeat, at predetermined time intervals, the preceding five steps, in order to assess over time the evolution of the immune-mediated disease in the patient.

v) additional “decision-maker” in the management of patients included in points i, ii, iii and iv.

It is clear that modifications and/or additions of parts and/or steps may be made to the use of the serological assay of the cytokine B-Lymphocyte stimulator in a diagnostic and prognostic method in the course of the immune-mediated diseases as described heretofore, without departing from the field and scope of the present invention. It is also clear that, although the present invention has been described with reference to specific examples, a skilled in the art shall be able to achieve other equivalent forms of the use of the serological assay of the cytokine B-Lymphocyte stimulator in diagnostic and prognostic method, having the characteristics as set forth in the claims and hence all coming within the field of protection defined thereby.

It will be appreciated by those skilled in the art that changes could be made to the embodiments described above without departing from the broad inventive concept thereof. It is understood, therefore, that this invention is not limited to the particular embodiments disclosed, but it is intended to cover modifications within the spirit and scope of the present invention as defined by the appended claims.

We claim:

1. A method of using an assay of cytokine B-lymphocyte Stimulator (BLyS) as a diagnostic marker for diagnosing a risk of an immune-mediated disease in a patient who is a candidate for blood transfusion or who underwent blood transfusion with or without symptoms or signs suggestive for transfusion reactions, the method comprising:
(a) taking a sample of blood from the patient;
(b) examining the blood sample to determine concentration of cytokine BLyS in the blood sample;
(c) comparing the concentration of cytokine BLyS determined in (b) and one or more reference values of concentration of cytokine BlyS previously obtained on a healthy control group;
(d) identifying a significant deviation, deriving from the comparison in (c), between the concentration of cytokine BLyS determined in (b) and the reference values of concentration of cytokine BLyS previously obtained on the healthy control group, so as to select the patient as affected by the immune-mediated disease;
(e) comparing the concentration of cytokine BLyS determined in (b) for the patient selected as affected by the immune-mediated disease and one or more reference values of concentration of cytokine BLyS in a range of BLyS reference values previously obtained on at least one of a series of patients with the immune-mediated disease or obtained on established diagnosis of the active disease or its particular course; and
(f) attributing an increased risk of developing a determinate immune-mediated disease, based on the comparison made in (e).

2. A method as in claim 1, wherein (b) is carried out using an automated apparatus based on an ELISA technique.

3. A method as in claim 1, wherein the immune-related disease is an immune-mediated disease related to blood transfusion.

4. A method as in claim 3, wherein the immune-mediated disease related to blood transfusion is selected from a disease attributable to transfusion from the group comprising a post-transfusion immunization, maternal-fetal incompatibility and a transfusion reaction.

5. A method as in claim 1, wherein the patient is a patient at risk of a transfusion reaction.

6. A method as in claim 5, wherein the transfusion reaction is an autoimmune disease or immunodeficiency.

7. A method as in claim 1, wherein the assay of cytokine BLyS is integrated by at least one of one examination or several examinations selected from the group comprising:
(i) physical and biochemical examination of the patient able to identify signs and symptoms of transfusion reactions and;
(ii) molecular analysis on DNA extracted by peripheral blood taken from the patient.

8. A method as in claim 7, wherein the signs and symptoms of transfusion reactions in (i) comprise alloantibodies and clinical manifestations, and wherein the molecular analysis on DNA extracted by peripheral blood taken from the patient in (ii) indicates a genetic predisposition linked or not linked to BLyS expression.

9. A method of using an assay of cytokine B-lymphocyte Stimulator (BLyS) as a prognostic marker for prognosis of an immune-mediated disease in a patient in need of a blood transfusion, the method comprising:
(a) taking a sample of blood from the patient;
(b) examining the blood sample to determine the concentration of cytokine BLyS in the blood sample;
(c) comparing the concentration of cytokine BLyS determined in (b) and one or more reference values of the concentration of cytokine BLyS;
(d) identifying a significant deviation, deriving from the comparison in (c), between the concentration of cytokine BLyS determined in (b) and the reference values considered in (c); and
(e) assigning a prognosis to the deviation identified in (d) with regard to the immune-mediated disease.

10. A method as in claim 9, wherein the immune-mediated disease is selected from a disease attributable to a transfusion from the group comprising a post-transfusion immunization, maternal-fetal incompatibility and a transfusion reaction.

11. A method as in claim 9, wherein the assay of cytokine BLyS is integrated by at least one of one examination or several examinations selected from the group comprising:
(i) physical and biochemical examination of the patient able to identify signs and symptoms of transfusion reactions and;
(ii) molecular analyses on DNA extracted by peripheral blood taken from the patient.

12. A method as in claim 11, wherein the signs and symptoms of transfusion reactions in (i) comprise alloantibodies and clinical manifestations, and wherein the molecular analysis on DNA extracted by peripheral blood taken from the patient in (ii) indicates a genetic predisposition linked or not linked to BLyS expression.

13. A method of using an assay of cytokine BLyS as a marker for monitoring treatment of a patient being treated for an immune-mediated disease, the method comprising:
(a) taking a sample of blood from the patient before at least one of a blood transfusion and initiation of a dedicated anti-BLyS therapy;
(b) examining the blood sample to determine the concentration of cytokine BLyS in the blood sample;
(c) taking one or more samples of blood from the patient at predefined time intervals from at least one of the respective blood transfusion and the initiation of the dedicated anti-BLyS therapy;
(d) examining the one or more blood samples of (c) to determine the concentration of cytokine BLyS in the one or more blood samples of (c);
(e) comparing the concentration of cytokine BLyS determined in the second step and the values of the concentration of cytokine BLyS detected in the patient in the fourth step;
(f) identifying a significant deviation, deriving from the comparison of (e), between the concentration of cytokine BLyS determined in (b) and the values of BLyS detected in (d); and
(g) attributing a risk of at least one of a transfusion reaction and effectiveness to the therapeutic treatment on the basis of the deviation identified in (f).

14. A method as in claim 13, wherein the immune-mediated disease is selected from a disease attributable to a transfusion from the group comprising a post-transfusion immunization, maternal-fetal incompatibility and a transfusion reaction.

15. A method as in claim 13, wherein the assay of cytokine BLyS is integrated by at least one of one examination or several examinations selected from the group comprising:
(i) physical and biochemical examination of the patient able to identify signs and symptoms of transfusion reactions and;
(ii) molecular analyses on DNA extracted by peripheral blood taken from the patient.

16. A method as in claim 15, wherein the signs and symptoms of transfusion reactions in (i) comprise alloantibodies and clinical manifestations, and wherein the molecular analysis on DNA extracted by peripheral blood taken from the patient in (ii) indicates a genetic predisposition linked or not linked to BLyS expression.
